(19) United States
(12) Patent Application Publication
(10) Pub. No.: US 2012/0149576 A1
(43) Pub. Date: Jun. 14, 2012

(54) METHOD FOR CULTIVATING CROP PLANT

(75) Inventors: Takanori Saijo, Tokyo (JP); Akitsu Nagasawa, Kobe-shi (JP)

(73) Assignee: SUMITOMO CHEMICAL COMPANY, LIMITED

(21) Appl. No.: 13/377,115

(22) PCT Filed: Jun. 9, 2010
(86) PCT No.: PCT/JP2010/060154

§ 371 Date: Feb. 21, 2012

(30) Foreign Application Priority Data
Jun. 11, 2009 (JP) 2009-140464

(51) Int. Cl. A01N 43/54; A01P 13/00; C12Q 1/26

(52) U.S. Cl. 504/243; 435/25

(57) ABSTRACT

The present invention enables providing a method for cultivating a crop plant into which has been introduced either one or both of (1) DNA comprising a nucleotide sequence encoding the amino acid sequence of a cytochrome P450 that shows saflufenacil metabolizing activity and (2) DNA comprising a nucleotide sequence encoding the amino acid sequence of a protein that shows protoporphyrinogen IX oxidase activity, wherein said method comprises applying a weed control agent that contains saflufenacil as an active ingredient to an area where said crop plant is cultivated; among others.

### TECHNICAL FIELD

The present invention relates to a method for cultivating a crop plant, and so on.

### BACKGROUND ART

Traditionally, weed control using weed control agents has been carried out for cultivation of agricultural crops. When selective weed control agents are used, more than one type of weed control agents are generally needed to be applied to cultivated agricultural crops. When non-selective weed control agents are used, they tend to show a high phytotoxic effect on crop plants, although they may reduce the labor and costs required to apply such a weed control agent.

Compounds that inhibit protoporphyrinogen IX oxidase (hereinafter may be referred to as “PPO”), which is involved in chlorophyll biosynthesis, are used as active ingredients of non-selective weed control agents. A variety of molecular species are known as compounds that inhibit protoporphyrinogen IX oxidase. As examples of such compounds, a group of compounds having a particular uracil-substituted phenyl sulfamoyl carboxamide structure are disclosed in WO01/83459.

To date, plants showing resistance to herbicidal compounds that are active ingredients of weed control agents, for example, plants showing resistance to protoporphyrinogen IX oxidase inhibitory-type herbicidal compounds, have been artificially generated.

Examples of plants known to be conferred artificially with resistance to a protoporphyrinogen IX oxidase inhibitory-type herbicidal compound (hereinafter, such a compound may be referred to as a “PPO inhibitor”; and such plants as “PPO inhibitor-resistant plants”) include:

(1) a plant overexpressing protoporphyrinogen IX oxidase in the plant;

(2) a plant expressing variant protoporphyrinogen IX oxidase into which a mutation that reduces the susceptibility to the herbicidal compound has been artificially introduced (see, for example, WO97/32011);

(3) a plant expressing a actinomyces cytochrome P450 capable of metabolizing and inactivating the herbicidal compound in the plant (see, for example, WO03/40370); and

(4) a plant into which (a) DNA having a nucleotide sequence encoding the amino acid sequence of a cytochrome P450 that shows activity of metabolizing the herbicidal compound and (b) DNA having a nucleotide sequence encoding the amino acid sequence of a protein that exhibits protoporphyrinogen IX oxidase activity have been introduced (see, for example, EP1598423).

### DISCLOSURE OF THE INVENTION

While effective weed control may be achieved by applying a PPO inhibitor to an area where PPO inhibitor-resistant plants are grown, there are cases where some combinations of a PPO inhibitor-resistant plant and a PPO inhibitor does not allow for increasing the amount of the PPO inhibitor used because of its phytotoxicity to the crop plant, resulting in an unsatisfactory weed control effect. Thus, the development of a preferred method for cultivating a PPO inhibitor-resistant plant by means of a combination with a particular PPO inhibitor is desired.

The present invention provides the following [1] to [10], among others.

[1]

A method for cultivating a crop plant into which has been introduced either one or both of:

(1) DNA comprising a nucleotide sequence encoding the amino acid sequence of a cytochrome P450 that shows saflufenacil metabolizing activity, and

(2) DNA comprising a nucleotide sequence encoding the amino acid sequence of a protein that shows protoporphyrinogen IX oxidase activity;

wherein said method comprises applying a weed control agent that contains saflufenacil as an active ingredient to an area where said crop plant is cultivated.

[2]

The cultivation method according to [1], wherein said cytochrome P450 is an actinomyces cytochrome P450.

[3]

The cultivation method according to [1], wherein said cytochrome P450 is selected from the group consisting of:

(1) cytochrome P450 derived from actinomyces belonging to the genus Streptomyces;

(2) cytochrome P450 comprising an amino acid sequence having 90% or more sequence homology with the amino acid sequence of SEQ ID NO: 1 or 2;

(3) cytochrome P450 comprising the amino acid sequence of SEQ ID NO: 1; and

(4) cytochrome P450 comprising the amino acid sequence of SEQ ID NO: 2.

[4]

The cultivation method according to [1], wherein said protein that shows protoporphyrinogen IX oxidase activity is derived from a plant.

[5]

The cultivation method according to [1], wherein said protein that shows protoporphyrinogen IX oxidase activity is selected from the group consisting of:

(1) a protein which is derived from a plant and shows protoporphyrinogen IX oxidase activity that is inhibited by saflufenacil;

(2) a protein which is derived from a plant and shows protoporphyrinogen IX oxidase activity that is not inhibited by saflufenacil;

(3) a protein comprising an amino acid sequence with 90% or more sequence homology with the amino acid sequence of SEQ ID NO: 3 and showing protoporphyrinogen IX oxidase activity; and

(4) a protein comprising the amino acid sequence of SEQ ID NO: 3 and showing protoporphyrinogen IX oxidase activity.

[6]

A method for screening a cell resistant to a protoporphyrinogen IX oxidase inhibitory-type herbicidal compound, wherein said method comprises:

introducing into and expressing in a plant cell either one or both of:

(1) DNA comprising a nucleotide sequence encoding the amino acid sequence of a cytochrome P450 that shows saflufenacil metabolizing activity, and

(2) DNA comprising a nucleotide sequence encoding the amino acid sequence of a protein that shows protoporphyrinogen IX oxidase activity; and

contacting said plant cell with saflufenacil.

[7]

The screening method according to [6], wherein said cytochrome P450 is an actinomyces cytochrome P450.

[8]

The screening method according to [6], wherein said cytochrome P450 is selected from the group consisting of:

(1) cytochrome P450 derived from actinomyces belonging to the genus Streptomyces;

(2) cytochrome P450 comprising an amino acid sequence having 90% or more sequence homology with the amino acid sequence of SEQ ID NO: 1 or 2;

(3) cytochrome P450 comprising the amino acid sequence of SEQ ID NO: 1; and

(4) cytochrome P450 comprising the amino acid sequence of SEQ ID NO: 2.

The screening method according to [6], wherein said protein that shows protoporphyrinogen IX oxidase activity is derived from a plant.

[10]

The screening method according to [6], wherein said protein that shows protoporphyrinogen IX oxidase activity is selected from the group consisting of:

(1) a protein which is derived from a plant and shows protoporphyrinogen IX oxidase activity that is inhibited by saflufenacil;

(2) a protein which is derived from a plant and shows protoporphyrinogen IX oxidase activity that is not inhibited by saflufenacil;

(3) a protein comprising an amino acid sequence with 90% or more sequence homology with the amino acid sequence of SEQ ID NO: 3 and showing protoporphyrinogen IX oxidase activity; and

(4) a protein comprising the amino acid sequence of SEQ ID NO: 3 and showing protoporphyrinogen IX oxidase activity.

The present invention provides a method for cultivating a PPO inhibitor-resistant plant, which method allows for increasing the amount of the PPO inhibitor used thereby affording a satisfactory weed control effect, among others.

### BRIEF DESCRIPTION OF THE DRAWINGS

FIG. 1 is a figure showing a photograph of leaf discs of a wild-type SR-1 (SR-1WT) plant and the recombinant soy lines 1609soy#25, P023 and P-6-1 after 8 days of static culture on MS agar medium supplemented with saflufenacil.

### MODE FOR CARRYING OUT THE INVENTION

The present invention is described in detail below.

The cultivation method of the present invention is a method for cultivating a crop plant into which has been introduced either one or both of;

(1) DNA comprising a nucleotide sequence encoding the amino acid sequence of a cytochrome P450 that shows saflufenacil metabolizing activity, and

(2) DNA comprising a nucleotide sequence encoding the amino acid sequence of a protein that shows protoporphyrinogen IX oxidase activity;

wherein said method comprises applying a weed control agent that contains saflufenacil as an active ingredient to an area where said crop plant is cultivated.

As used in the present invention, the “weed control agent” refers to a composition containing saflufenacil as an active ingredient.

Saflufenacil (IUPAC Name: N′-{2-chloro-4-fluoro-5-[1,2,3,6-tetrahydro-3-methyl-2,6-dioxo-4-(trifluoromethyl)pyrimidine-1-yl]benzoyl}-N-isopropyl-N-methyl sulfamide) (CAS Registry Number: 372137-35-4) is a known compound having protoporphyrinogen IX oxidase inhibitory-type herbicidal activity and being described, for example, in WO01/83459.

There is no particular limitation on the saflufenacil content in the weed control agent, as long as it shows the effect of the present invention.

The weed control agent may contain, if necessary, other herbicidal compounds, insecticidal compounds, fungicidal compounds, plant growth regulatory compounds, fertilizer ingredients, and additives widely used in weed control agents, in addition to saflufenacil as an active ingredient. Examples of the herbicidal compounds include [(phosphonomethyl)amino]acetic acid (CAS Registry Number: 1071-83-6).

Crop plants that can be cultivated using the cultivation method of the present invention are crop plants into which has been introduced either one or both of:

(1) DNA comprising a nucleotide sequence encoding the amino acid sequence of a cytochrome P450 that shows saflufenacil metabolizing activity; and

(2) DNA comprising a nucleotide sequence encoding the amino acid sequence of a protein that shows protoporphyrinogen IX oxidase activity.

Such crop plants and method for their preparation are described in detail below.

As used herein, the term “cytochrome P450” has a usual meaning; it refers to a group of protohem-containing proteins that have been designated on the basis of their spectroscopic properties that when bound to carbon monoxide under reducing conditions, these proteins show a Soret absorption band at wavelengths near 450 nm.

As used herein, the phrase “show saflufenacil metabolizing activity” means that the cytochrome P450 has ability to cause saflufenacil to undergo monooxygenation and subsequent detachment of functional groups.

The cytochrome P450 may be of:

(1) a type that receives electrons through electron transfer with both ferredoxin and NADPH-ferredoxin reductase; or

(2) a type that receives electrons directly from NADPH-cytochrome P450 reductase. Preferably, it may be of the former type.

In the cells of a crop plant that can be cultivated using the cultivation method of the present invention, i.e., in the cells of a transgenic host, the cytochrome P450 may be present in any intracellular organelles in the host cell or in the cytoplasm. The ferredoxin may be endogenous ferredoxin in the host cells or heterologous ferredoxin introduced exogenously into the host cells.

The location of a gene which has a nucleotide sequence encoding the amino acid sequence of the cytochrome P450 and which has been introduced into the host cell, for example, by a method as described below may be in any of the intracellular organelles in the cell or in the chromosomes in the nucleus. The location of the cytochrome P450 may be in any of the intracellular organelles in the cell, in the cytoplasma, or in the extracellular space. Preferably it may be in an intracellular organelle, and more preferably in chloroplasts in the host cell.

In order for cytochrome P450 to be transported into an intracellular organelle in the cell, for example, it may be introduced into the host cell a chimeric DNA constructed by linking in frame a DNA having a nucleotide sequence encoding an intracellular organelle transit signal sequence to upstream of a DNA having a nucleotide sequence encoding the amino acid sequence of the cytochrome P450. In this context, the phrase “linked in-frame” refers to a state in which the open reading frame of the nucleotide sequence encoding an intracellular organelle transit signal sequence is linked to the open reading frame of the nucleotide sequence encoding the amino acid sequence of the cytochrome P450 such that a continuous open reading frame can be formed. Examples of the transit signal sequences that lead to transportation and localization of proteins into intracellular organelles in the host cell may include the transit signal sequence described in U.S. Pat. No. 5,717,084, which is derived from a cytoplasmatic precursor protein of a protein localized in plant chloroplasts, and chimeric sequences as described in USRE36449, which are comprised of a plurality of transit signal sequences. More specifically, examples of the signal sequences include a chloroplast transit signal peptide derived from the small subunit of soybean ribulose-1,5-bisphosphate carboxylase (hereinafter may be referred to as RuBPCO), which is obtainable using the method described in WO03/040370.

There is no particular limitation on the source of the cytochrome P450; it may be derived from any biological source, such as animal tissue, plant tissue, fungi, yeast and bacteria. A preferred example may be an actinomyces cytochrome P450. As used herein, “actinomyces” refers to a group of prokaryotes belonging to the order Actinomycetales, which are gram-positive bacteria, divided into eight genera, including Streptomyces, Actinomyces, Mycobacterium, Frankia and Nocardia. More preferred cytochrome P450 may be cytochrome P450 derived from actinomyces belonging to the genus Streptomyces, specifically, cytochrome P450 derived from Streptomyces phaeochromogenes, Streptomyces testaceus, Streptomyces achromogenes, Streptomyces griseofuscus, Streptomyces thermocoerulescens, Streptomyces nogalater, Streptomyces tsusimaensis, Streptomyces glomerochromogenes, Streptomyces olivochromogenes, Streptomyces ornatus, Streptomyces griseus, Streptomyces lanatus, Streptomyces misawanensis, Streptomyces pallidus, Streptomyces roseorubens, Streptomyces rutgersensis, Streptomyces steffisburgensis, or Saccharopolyspora taberi.

A gene (DNA) having a nucleotide sequence encoding the amino acid sequence of such a cytochrome P450 may be a gene having a naturally occurring nucleotide sequence, or a gene modified to optimal codons for the host. Also, it may be a gene encoding the amino acid sequence of a naturally occurring cytochrome P450 into which substitution, addition or deletion of one or more amino acid residues has been introduced. Specifically, examples of genes encoding cytochrome P450 include a gene encoding cytochrome P450 described in WO03/040370.

As used herein, gene (DNA) refers to a DNA segment involved in the production of the polypeptide chain, and may or may not contain upstream and downstream regions (e.g. a leader sequence), and an intervening sequence (intron).

Examples of techniques for artificially deleting, adding or substituting amino acid residues in the amino acid sequence include methods for introducing site-directed mutations into DNA having a nucleotide sequence encoding an amino acid sequence. Specifically, examples of such methods include a method utilizing amber mutation (the gapped duplex method, Nucleic Acids Res., 12, 9441-9456 (1984)) and a method using PCR with primers for introducing mutations. The examples also include methods for randomly introducing mutations into DNA having a nucleotide sequence encoding any one of the amino acid sequences. Specifically, the examples include a method of performing PCR using, as a template, DNA having a nucleotide sequence encoding an amino acid sequence and using a primer pair that can amplify the full length DNA under reaction conditions with different concentrations of each of dATP, dTTP, dGTP and dCTP used as a substrate, or with an increased concentration of Mg²⁺ for promoting the polymerase reaction.

Examples of such PCR methods include the conventional method described in Method in Molecular Biology, (31), 1994, 97-112.

Particularly preferably, the cytochrome P450 described above may be:

(1) cytochrome P450 comprising the amino acid sequence of SEQ ID NO: 1;

(2) cytochrome P450 comprising the amino acid sequence of SEQ ID NO: 2; or

(3) cytochrome P450 comprising an amino acid sequence substantially identical to the amino acid sequence of SEQ ID NO: 1 or 2.

The amino acid sequences of SEQ ID NOs: 1 and 2 are those of actinomyces cytochrome P450.

As used herein, “substantially identical amino acid sequence” refers to an amino sequence having a sequence homology of preferably 90% or more, more preferably 95% or more, and even more preferably 98% or more.

The “sequence homology” of an amino acid sequence is herein determined by comparing two optimally aligned sequences over the entire region of the reference amino acid sequence. In this context, in order to optimally align the reference amino acid sequence, additions or deletions (e.g. gaps) may be allowed. Such sequence homology may be calculated based on alignment by homology analysis using a program, such as FASTA (Pearson & Lipman, Proc. Natl. Acad. Sci. USA, 4, 2444-2448 (1988)), BLAST (Altschul at al., Journal of Molecular Biology, 215, 403-410 (1990)) and CLUSTAL W (Thompson, Higgins and Gibson, Nucleic Acid Research, 22, 4673-4680 (1994a)). These programs are publicly available, for example, from the Web page (http://www.ddbj.nig.ac.jp/Welcome-e.html) of DNA Data Bank of Japan (the international DNA data bank administered by Center for Information Biology and DNA Data Bank of Japan; CIB/DDBJ). Sequence homology may also be calculated using a commercially available sequence analysis software program. Specifically, sequence homology can be calculated by aligning sequences by homology analysis according to the Lipman-Pearson method (Lipman, D. J. and Pearson, W. R., Science, 227, 1435-1441, (1985)) using GENETYX-WIN Ver. 6 (GENETYX Corporation). For example, the result that a 90% sequence homology is found between the amino acid sequences of SEQ ID NOs: 1 and 2 is calculated by homology analysis to align the sequences.

As used herein, the “substantially identical amino acid sequence” refers to

(1) an amino acid sequence having from 1 to 7, preferably from 1 to 5, and more preferably from 1 to 3 amino acid deletions in the reference amino acid sequence;

(2) an amino acid sequence having from 1 to 20, preferably from 1 to 15, and more preferably from 1 to 10 amino acid additions (or insertions) in the reference amino acid sequence;

(3) an amino acid sequence in which from 1 to 7, preferably from 1 to 5, and more preferably from 1 to 3 amino acids are substituted with other amino acids; and

(4) an amino acid sequence having a combination of these deletions, additions and substitutions.

As used herein, “a protein that shows protoporphyrinogen IX oxidase activity” refers to a protein that has enzymatic activity that produces protoporphyrin IX by oxidation of protoporphyrinogen IX in the cell.

The “protein that shows protoporphyrinogen IX oxidase activity” may be a protein whose activity is inhibited by saflufenacil, an inhibitor of protoporphyrinogen IX oxidase activity (hereinafter, may be referred to as “PPO activity”), or a protein whose activity is not inhibited by saflufenacil.

Examples of a protein whose PPO activity is inhibited by saflufenacil include proteins possessed by plant species that exhibit growth inhibition, such as chlorosis, lesions or death, in response to a normal dose of saflufenacil. In contrast, examples of a protein whose PPO activity is not inhibited by saflufenacil include proteins possessed by plant species that exhibit resistance to normal doses of herbicides, proteins possessed by animals, and proteins possessed by microorganisms.

There is no particular limitation on the origin of the “protein that shows protoporphyrinogen IX oxidase activity”. It may be a protein derived, for example, from Esherichia coil (Genebank accession X68660), Bacillus subtilis (Genebank accession M97208), Haemophilus influnzae (Genebank accession L42023), mouse (Genebank accession D45185), human (Genebank accession D35537), Arabidopsis thaliana (Genebank accession D83139), or tobacco (Genebank accessions Y13465 and Y13466). Inter alia, it may be a protein of plant origin (e.g. Arabidopsis thaliana (Genebank accession D83139), or tobacco (Genebank accessions Y13465 and Y13466)).

A gene (DNA) having a nucleotide sequence encoding the amino acid sequence of the protein showing PPO activity may be a gene having a naturally occurring sequence, or may be a gene which has modifications to optimal codons for the host. Also, such a gene may be a gene encoding the amino acid sequence of a naturally occurring protein showing PPO activity into which substitution, addition or deletion of one or more amino acid residues has been introduced. Specifically, examples of genes encoding the amino acid sequences of proteins showing PPO activity, in particular, genes encoding the amino acid sequences of mutant PPOs whose PPO activity is not inhibited by saflufenacil, include the genes described in WO1995/034659, WO1997/032011 or WO1997/004089.

Examples of techniques for artificially deleting, adding or substituting amino acid residues in the amino acid sequences include those described above for cytochrome P450.

Particularly preferably, the protein of the present invention that shows protoporphyrinogen IX oxidase activity may be:

(1) a protein comprising the amino acid sequence of SEQ ID NO: 3 and showing protoporphyrinogen IX oxidase activity; or

(2) protoporphyrinogen IX oxidase comprising an amino acid sequence substantially identical to the amino acid sequence of SEQ ID NO: 3.

SEQ ID NO: 3 shows the amino acid sequence of a soybean protoporphyrinogen IX oxidase variant.

Any conventional gene transfer method may be used as a method for preparing a crop plant that can be cultivated using the method of the present invention by introducing, into the crop plant, DNA having a nucleotide sequence encoding the amino acid sequence of the cytochrome P450, and DNA having a nucleotide sequence encoding the amino acid sequence of the protein that shows PPO activity. Specifically, for example, chmeric DNA as described above for P450 or DNA constructed by operably linking such chimeric DNA to a promoter operable in the host cells may be incorporated into a vector available in the plant from which the host cells are obtained, and then the vector may be introduced into the host cells. When a vector plasmid that already contains a promoter operable in the host cells is used, the chimeric DNA may be inserted downstream of the promoter so that the promoter contained by the vector plasmid and the chimeric DNA can be linked in an operable manner.

In this context, a promoter operable in host cells, such as plant cells, refers to a nucleotide sequence that is attached upstream of the 5′-end of the nucleotide sequence of a gene (structural gene) having a nucleotide sequence encoding the amino acid sequence of a protein to be introduced into host cells, such as plant cells, and capable of allowing transcription of RNA containing the structural gene. Examples of promoters operable in host cells, such as plant cells, include T-DNA-derived constitutive promoters, such as the nopaline synthase gene (NOS) promoter and the octopine synthase gene (OCS) promoter; plant virus-derived promoters, such as the cauliflower mosaic virus 19S or 35S promoter; inducible promoters, such as the phenylalanine ammonialyase gene promoters, the chalcone synthase gene promoters and pathogenesis-related protein gene promoters, or the synthesized chimeric promoters described in WO2008/111661; and the plant promoters described in WO2000/020613. In addition, a terminator operable in host cells, such as plant cells, may be linked downstream of DNA comprising a promoter operable in host cells such as plant cells, as described above, and a nucleotide sequence encoding the amino acid sequence of a protein exhibiting PPO activity or of cytochrome P450.

A terminator operable in host cells, such as plant cells, refers to a nucleotide sequence that is attached downstream of the 3′-end of the nucleotide sequence of a gene (structural gene) having a nucleotide sequence encoding the amino acid sequence of a protein to be introduced into host cells, such as plant cells, and capable of allowing addition of a polyadenine sequence for stabilizing of the transcription of RNA containing the structural gene. Examples of terminators operable in plant cells include T-DNA-derived constitutive terminators, such as the nopaline synthase gene (NOS) terminator; plant virus-derived terminators, such as the garlic virus GV1 and GV2 promoters; and the plant terminators described in WO2000/020613.

Examples of crop plants that can be cultivated using the cultivation method of the present invention include soybean, pea, kidney bean, alfalfa, Lotus japonicus, clover, peanut, sweet pea, walnut, tea, cotton, pepper, cucumber, water melon, pumpkin, melon, Japanese radish, rapeseed, canola, sugar beet, lettuce, cabbage, broccoli, cauliflower, Arabidopsis thaliana, tobacco, eggplant, potato, sweet potato, taro, Jerusalem artichoke, tomato, spinach, asparagus, carrot, flax, sesame, endive, chrysanthemum, geranium, antirrhinum, carnation, dianthus, periwinkle, bouvardia, gypsophila, gerbera, Eustoma grandiflorum, tulip, stock, Limonium, cyclamen, saxifrage, swamp chrysanthemum, violet, rose, cherry, apple, pear, grape, strawberry, Japanese plum, almond, orange, lemon, banana, mango, papaya, kiwi, coffee, Japanese quince, Rhododendron indicum, azalea, poinsettia, Barbados nut, cassava, oil palm, coconut palm, olive, gentian, cosmos, morning-glory, sunflower, ginkgo, Japanese cedar, Japanese cypress, poplar, pine, sequoia, oak, willow, eucalyptus, kenaf, water lily, Eucommia ulmoides, beech, Castor bean, bamboo, sugar cane, rice, wheat, barley, rye, oat, maize, sorghum, lawn grass, tall fescue, switchgrass, Japanese silver grass, green onion, onion, garlic, lily, Tiger lily, orchid, gladiolus and pineapple.

Plant cells from variety of plant materials, such as plant tissue, individual plants, cultured cells and seed may be used as host cells.

Examples of methods for introducing DNA having the structural gene to which has been linked a promoter and a terminator operable in host cells such as plant cells include the Agrobacterium infection method (Japanese Patent Publication No H02-58917 and Japanese Patent Application Publication No. S60-70080), electroporation into protoplasts (Japanese Patent Application Publication Nos. S60-251887 and H05-68575) and the particle gun method (Japanese Patent Application Publication Nos. H05-508316 and 863-258525).

In this process, for example, a selectable marker gene selected from among a hygromycin phosphotransferase gene, neomycin phosphotransferase gene, chloramphenicol acetyltransferase gene, and so on, and DNA having a nucleotide sequence encoding the amino acid sequence of a protein exhibiting PPO activity or DNA having a nucleotide sequence encoding the amino acid sequence of a cytochrome P450 that shows activity of metabolizing the herbicidal compound saflufenacil can be cotransfected into host cells, such as plant cells, and resulting transfectants can be selected using, for example, the phenotype of the marker gene as an indicator. The marker gene may be integrated into the same vector for transfection in tandem with DNA having a nucleotide sequence encoding the amino acid sequence of a protein showing PPO activity or DNA having a nucleotide sequence encoding the amino acid sequence of a cytochrome P450 that shows activity of metabolizing the herbicidal compound saflufenacil.

Alternatively, a vector plasmid containing the selectable marker may be co-transfected with DNA having a nucleotide sequence encoding the amino acid sequence of a protein that shows PPO activity or DNA having a nucleotide sequence encoding the amino acid sequence of a cytochrome P450 that shows activity of metabolizing the herbicidal compound saflufenacil.

Transfectants in which the gene of interest has been introduced may he selected by culturing plant cells in which a vector containing a gene of interest has been introduced on the medium supplemented with the herbicidal compound saflufenacil and then by isolating clones capable of growing on the medium; that is, saflufenacil may be used to screen for cells resistant to a protoporphyrinogen IX oxidase inhibitory-type herbicidal compound. The concentration of saflufenacil added to the medium ranges, for example, from 0.001 to 1.0 ppm, and preferably from 0.01 to 0.1 ppm. The resistance can be monitored by observing the growth of the cells cultured under light conditions in the medium supplemented with saflufenacil after 2 to 3 subcultures every 1 to 2 weeks.

The method of screening for cells resistant to a protoporphyrinogen IX oxidase inhibitory-type herbicidal compound (e.g. saflufenacil) by using saflufenacil is also an aspect of the present invention.

The method for introducing into a plant both DNA having a nucleotide sequence encoding the amino acid sequence of a protein that shows PPO activity and DNA having a nucleotide sequence encoding the amino acid sequence of a cytochrome P450 that shows saflufenacil metabolizing activity may be either (1) a method in which both the DNAs are mixed for simultaneous introduction into the same plant cell, or (2) a method in which DNA comprising a tandem linkage of the DNAs is introduced into the same plant cell.

The retention of both the DNAs by the transfectants can be verified by performing analysis of the DNA prepared from the transfectants by using, for example, genetic engineering techniques (confirmation of restriction sites, nucleotide sequence analysis, southern hybridization, PCR, etc.), as described, for example, in “Molecular Cloning: A Laboratory Manual,” 2nd edition (1989), Cold Spring Harbor Laboratory Press.

Specifically, for example, according to the method described in Experimental Protocols for model plants: Rice and Arabidopsis (Edited by Shimamoto K., and Okada K., 1996, SHUJUNSHA, Co., Ltd), Chapter 4, a rice or Arabidopsis plant into which both or either of the DNAs has been introduced may be obtained. Also, according to the method described in Japanese Patent Application Publication NO. H03-291501, a soybean plant into which both or either of the DNAs has been introduced may be obtained by introduction into soybean adventitious embryos using a particle gun. Similarly, according to the method described in Fromm, M. E., at al., Bio/Technology, 8; p 838 (1990), a maize plant into which both or either of the DNAs has been introduced may be obtained by introduction into maize adventitious embryos using a particle gun. Similarly, according to the method described in Takumi, et al., Japanese Journal of Breeding, 1995, vol. 44, additional vol. 1, p. 57, a wheat plant into which both or either of the DNAs has been introduced may be obtained by introduction into immature wheat scutellum cultured under sterile conditions by using a particle gun. Similarly, according to the method described in Hagia et el., Japanese Journal of Breeding, 1995, vol. 44, additional vol. 1, p. 67, a barley plant into which both or either of the DNAs has been introduced may be obtained by introduction into immature barley scutellum cultured under sterile conditions by using a particle gun.

From the transfectants thus obtained, plants may be regenerated according to the plant cell culture method described, for example, in “Plant Cell and Tissue Culture: Practice, application and prospects,” (Edited by Harada and Komamine, Rikougakusya, 1979), pp. 65-118, thereby obtaining a transgenic plant into which both or either of the DNAs has been introduced.

Furthermore, it is also possible to obtain a plant of desired variety into which both or either of the DNAs has been introduced, by introducing both or either of the DNAs into the genome of a plant of the desired variety by crossing a transgenic plant into which both or either of the DNAs has been introduced and which expresses the DNAs with the plant of the desired variety.

It is further possible to generate a plant used in the present invention by introducing separately DNA having a nucleotide sequence encoding the amino acid sequence of a protein exhibiting PPO activity and DNA having a nucleotide sequence encoding the amino acid sequence of a cytochrome P450 that exhibits activity of metabolizing the herbicidal compound saflufenacil into plant cells, and screening and regenerating them to form individual plants, followed by crossing between progeny lines of the regenerated transfectants.

Specific crop plants are described in detail below.

For example, in order to produce recombinant soybean lines in which has been introduced a DNA fragment having a nucleotide sequence encoding the amino acid sequence of mutant soybean PPO, a DNA fragment having a nucleotide sequence encoding the amino acid sequence of SEQ ID NO: 1, a DNA fragment having a nucleotide sequence encoding the amino acid sequence of SEQ ID NO: 2, or a DNA fragment having a nucleotide sequence encoding the amino acid sequence of SEQ ID NO: 3, these DNA fragments are independently introduced into soybean adventitious embryos using a particle gun, according to the method described in Japanese Patent Application Publication NO. H03-291501. Subsequently, the recombinant soybean lines thus obtained are crossed to obtain crossed lines. To investigate the resistance of the recombinant soybean lines or crossed lines to the herbicidal compound saflufenacil, assessment by scoring susceptibility in an application test of the herbicidal compound saflufenacil may be conducted according to the method described below, for example, in Example 2 (for assessment by score indexes based on the degrees of adverse effects, such as death of individual plants and browning/chlorosis of leaves or stems, caused by application of a chemical solution).

For example, in order to produce recombinant maize lines in which has been introduced a DNA fragment having a nucleotide sequence encoding the amino acid sequence of mutant maize PPO, a DNA fragment having a nucleotide sequence encoding the amino acid sequence of SEQ ID NO: 1, a DNA fragment having a nucleotide sequence encoding the amino acid sequence of SEQ ID NO: 2, or a DNA fragment having a nucleotide sequence encoding the amino acid sequence of SEQ ID NO: 3, these DNA fragments are independently introduced into maize adventitious embryos using a particle gun, according to the method described in Fromm, M. E., et al., Bio/Technology, 8; p 838 (1990). Subsequently, the recombinant maize lines thus obtained are crossed to obtain crossed lines. To investigate the resistance of the recombinant maize lines or crossed lines to the herbicidal compound saflufenacil, assessment by scoring susceptibility in an application test of the herbicidal compound saflufenacil may be conducted according to the method described below, for example, in Example 2 (for assessment by score indexes based on the degrees of adverse effects, such as death of individual plants and browning/chlorosis of leaves or stems, caused by application of a chemical solution).

For example, in order to produce recombinant cotton lines in which has been introduced a DNA fragment having a nucleotide sequence encoding the amino acid sequence of mutant cotton PPO, a DNA fragment having a nucleotide sequence encoding the amino acid sequence of SEQ ID NO: 1, a DNA fragment having a nucleotide sequence encoding the amino acid sequence of SEQ ID NO: 2, or a DNA fragment having a nucleotide sequence encoding the amino acid sequence of SEQ ID NO: 3, these DNA fragments are independently introduced into cotton using the Agrobacterium method. Subsequently, the recombinant cotton lines thus obtained are crossed to obtain crossed lines. To investigate the resistance of the recombinant cotton lines or crossed lines to the herbicidal compound saflufenacil, assessment by scaring susceptibility in an application test of the herbicidal compound saflufenacil may be conducted according to the method described below, for example, in Example 2 (for assessment by score indexes based on the degrees of adverse effects, such as death of individual plants and browning/chlorosis of leaves or stems, caused by application of a chemical solution).

For example, in order to produce recombinant rapeseed lines in which has been introduced a DNA fragment having a nucleotide sequence encoding the amino acid sequence of mutant rapeseed PPO, a DNA fragment having a nucleotide sequence encoding the amino acid sequence of SEQ ID NO: 1, a DNA fragment having a nucleotide sequence encoding the amino acid sequence of SEQ ID NO: 2, or a DNA fragment having a nucleotide sequence encoding the amino acid sequence of SEQ ID NO: 3, these DNA fragments are independently introduced into rapeseed using electroporation. Subsequently, the recombinant rapeseed lines thus obtained are crossed to obtain crossed lines. To investigate the resistance of the recombinant rapeseed lines or crossed lines to the herbicidal compound saflufenacil, assessment by scoring susceptibility in an application test of the herbicidal compound saflufenacil may be conducted according to the method described below, for example, in Example 2 (for assessment by score indexes based on the degrees of adverse effects, such as death of individual plants and browning/chlorosis of leaves or stems, caused by application of a chemical solution).

For example, in order to produce recombinant wheat lines in which has been introduced a DNA fragment having a nucleotide sequence encoding the amino acid sequence of mutant cotton PPO, a DNA fragment having a nucleotide sequence encoding the amino acid sequence of SEQ ID NO: 1, a DNA fragment having a nucleotide sequence encoding the amino acid sequence of SEQ ID NO: 2, or a DNA fragment having a nucleotide sequence encoding the amino acid sequence of SEQ ID NO: 3, these DNA fragments are independently introduced into immature wheat scutellum using a particle gun, according to the method described in Takumi, et al., Japanese Journal of Breeding, 1995, vol. 44, additional vol. 1, p. 57. Subsequently, the recombinant wheat lines thus obtained are crossed to obtain crossed lines. To investigate the resistance of the recombinant wheat lines or crossed lines to the herbicidal compound saflufenacil, assessment by scoring susceptibility in an application test of the herbicidal compound saflufenacil may be conducted according to the method described below, for example, in Example 2 (for assessment by score indexes based an the degrees of adverse effects, such as death of individual plants and browning/chlorosis of leaves or stems, caused by application of a chemical solution).

In the cultivation method of the present invention, a weed control agent containing saflufenacil as a major ingredient is applied to the area where the plants according to the cultivation method of the present invention are grown. The application may be conducted, for example, by spraying the weed control agent to the area. Although the amount of application of the weed control agent may be appropriately determined depending, for example, on the application time and the types of weed, preferably, 1 to 1000 g per hectare of saflufenacil is applied. More preferably, 18 to 125 g per hectare of saflufenacil is applied.

There is no limitation on the method for the application. Any conventional method suitable for the form of the weed control agent may be employed.

The cultivation method of the present invention may preferably be applied to a wide range of crop plants as long as the plants are introduced with either one or both of DNA as defined below in (1) and (2) and they show saflufenacil resistance;

(1) DNA comprising a nucleotide sequence encoding the amino acid sequence of a cytochrome P450 that shows saflufenacil metabolizing activity; and

(2) DNA comprising a nucleotide sequence encoding the amino acid sequence of a protein that shows protoporphyrinogen IX oxidase activity.

Thus, the plants that can be cultivated using the cultivation method of the present invention may express either a protein exhibiting PPO activity or a cytochrome P450 that exhibits activity of metabolizing the herbicidal compound saflufenacil. More than one species of either of the proteins may be co-expressed, each one species of both the proteins may be co-expressed, or one species of either of the proteins and more than one species of the other may be co-expressed. In the plants according to the cultivation method of the present invention, the herbicidal compound saflufenacil is rapidly metabolized in vivo to compounds with lower herbicidal activity, or the herbicidal compound saflufenacil is unable to inhibit the PPO activity of the plants. As a result, application of the herbicidal compound saflufenacil may result in reduction in the adverse effects of the herbicide. Therefore, the plants according to the cultivation method of the present invention may show good growth even when saflufenacil are applied or added to the area where the plants that can be cultivated using the cultivation method of the present invention are cultivated or cultured.

The cultivation method of the present invention allows efficient elimination of plants, such as weeds, that are not the crop plants cultivated, and allows for improvement of the yield and quality, reduction in the amount of weed control agents used, and laborsaving.

### EXAMPLES

The present invention is specifically illustrated in detail below with reference to Examples, but it is not limited by these examples.

The DNAs introduced into the plants used in the following examples are shown below:

1609soy#17: DNA encoding the amino acid sequence of SEQ ID NO:1;

1609soy#25: DNA encoding the amino acid sequence of SEQ ID NO:1;

P023: DNA encoding the amino acid sequence of SEQ ID NO:3;

35S-2: DNA encoding the amino acid sequence of SEQ NO:3;

P-6-1; DNA encoding the amino acid sequence of SEQ ID NO:1 and DNA encoding the amino acid sequence of SEQ ID NO:3;

1584soy#16: DNA encoding the amino acid sequence of SEQ ID NO:2;

J16: DNA encoding the amino acid sequence of SEQ ID NO:1;

J18: DNA encoding the amino acid sequence of SEQ ID NO:1;

J26: DNA encoding the amino acid sequence of SEQ ID NO:1; and

J26: DNA encoding the amino acid sequence of SEQ ID NO:1.

### Example 1

### Resistance Test Using Leaf Discs

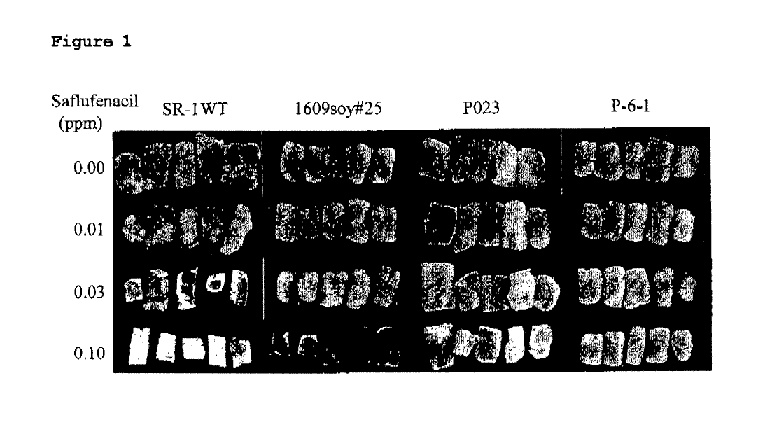

Homozygote seeds of the recombinant tobacco lines 1609soy#25 and P023, which are described in EP1598423, and progeny seeds of the recombinant tobacco line P-6-1, which is also described in EP1598423, were aseptically sown on MS agar medium supplemented with 100 mg/L kanamycin and screened for plants with kanamaycin resistance. Leaf discs were prepared by cutting true leaves of the selected plants at 1 month after sowing, and placed on MS agar medium supplemented with 0.01 ppm, 0.03 ppm or 0.10 ppm saflufenacil. As negative control, seeds of wild-type SR-1 tobacco plants were aseptically sown on MS agar medium and, similarly, leaf discs were prepared by cutting true leaves of the plants at 1 month after sowing, and placed on MS agar medium supplemented with 0.01 ppm, 0.03 ppm or 0.10 ppm saflufenacil. Then, the discs were observed at day 8. A photograph showing the results are shown in FIG. 1. In the wild-type SR-1 tobacco leaves, partial browning emerged at 0.01 ppm and overall chlorosis was observed at 0.10 ppm. In contrast, in the recombinant tobacco line 1609soy#25, no adverse effect was observed at 0.01 ppm, while partial browning and overall browning emerged at 0.03 ppm and 0.10 ppm, respectively. In the recombinant tobacco line P023, no adverse effect was observed up to 0.03 ppm and partial chlorosis was observed at 0.10 ppm. Further, in the progeny plants of the recombinant tobacco line P-6-1, no adverse effect was observed at the concentration of 0.10 ppm.

The leaf discs were prepared from 5 individual plants for each experimental plot. Scoring evaluation was conducted according to the degrees of adverse effects manifested as browning or chlorosis. The results are shown in Table 1. The scoring criteria are as follows:

“0”: Overall chlorosis of the leaf disc;

“1”: Overall browning of the leaf disc;

“2”: Partial browning or chlorosis of the leaf disc; and

“3”: No adverse effect on the leaf disc.

TABLE 1

| Saflufenacil | | | | |
|---|---|---|---|---|
| (ppm) | Wild-type SR-1 | 1609soy#25 | P023 | P-6-1 |
| 0.00 | 3.0 | 3.0 | 3.0 | 3.0 |
| 0.01 | 2.0 | 3.0 | 3.0 | 3.0 |
| 0.03 | 1.0 | 2.0 | 3.0 | 2.6 |
| 0.10 | 0.2 | 1.0 | 2.0 | 3.0 |

In the photograph shown in FIG. 1, the green-colored portions were color extracted using the image analysis software “Win ROOF ver.6.1.0” (MITANI Corporation) and their area was quantified. This was then divided by the area calculated by subtracting the area of the medium from the entire area of the image, thereby calculating the percentage of the area of the green-colored portions to the area of the leaf disc. The results are shown in Table 2.

TABLE 2

| | |
|---|---|
| Saflufenacil | Ratio of green area to leaf disc area (%) |

| (ppm) | Wild-type SR-1 | 1609soy#25 | P023 | P-6-1 |
|---|---|---|---|---|
| 0.00 | 85 | 86 | 89 | 89 |
| 0.01 | 77 | 85 | 89 | 87 |
| 0.03 | 20 | 80 | 86 | 78 |
| 0.10 | 2 | 8 | 51 | 83 |

### Example 2

### Resistance Test Using a Spreader: Tobacco

Homozygote seeds of the recombinant tobacco lines 1609soy#17 and #25, 1584soy#16, P023, and 35S-2, which are described in EP1598423, are aseptically sown on MS agar medium. Subsequently, the germinated individuals are transferred to culture pots charged with Kureha soil (Kureha Chemicals) and acclimated to the outdoor environment in the phytotron. They are than grown for two weeks in the phytotron. As negative control, seeds of wild-type SR-1 tobacco plants are aseptically sown on MS agar medium and, similarly grown in the phytotron. The plants thus obtained are subjected to the saflufenacil application test. The spray solution is prepared by dissolving saflufenacil in an appropriate solvent, adding an adjuvant, and diluting it with water.

Application of the spray solution to the plants are conducted using an wheeled automatic spreader (Mamba Architect Office), by homogenously spraying 20 mL of the solution onto the seedlings of the recombinant and wild-type tobacco plants placed within a 0.9 square-meter area to which the solution is applied. About two weeks later, the susceptibility of the tested recombinant tobacco plants to saflufenacil is compared with that of the wild-type SR-1 tobacco plants to saflufenacil. The results show that the homozygotes of the recombinant tobacco lines 1609soy#17 and #25, 1584soy#16, P023, and 35S-2 have lower susceptibility to saflufenacil as compared with the wild-type SR-1 tobacco plants; the recombinant lines exhibit saflufenacil resistance.

Scoring evaluation is conducted according to the degrees of adverse effects manifested as death of individual plants, or browning or chlorosis of the leaves or stems. The scoring criteria are as follows:

“0”: Death of the individual plant;

“1”: An adverse effect of browing or chlorosis emerges in the leaves or stems, and the plant growth is largely retarded; however, the plant is still alive;

“2”: An adverse effect of brewing or chlorosis emerges in the leaves or stems; however, the plant growth is not retarded and the plant is alive; and

“3”: An adverse effect of browing or chlorosis emerges slightly or almost invisible.

### Example 3

### Resistance Test Using a Spreader: Soybean

Seeds of the T2 generation of the recombinant soybean lines J16, J10, J26 and J28, which are described in US20060009361, are aseptically sown in the culture pots charged with Kureha soil and grown for about 3 weeks in the phytotron. As negative control, seeds of a wild-type soybean (cv. Jack) are similarly grown in the phytotron. The plants thus obtained are subjected to the saflufenacil application test. The application test is conducted according to the method described above in Example 2. About two weeks later, the susceptibility of the tested recombinant soybean plants to saflufenacil is compared with that of the wild-type soybean plants (cv. Jack) to saflufenacil. The results show that the recombinant soybean lines J16, J18, J26 and J28 have lower susceptibility to saflufenacil as compared with the wild-type soybean (cv. Jack); the recombinant lines exhibit saflufenacil resistance.

### INDUSTRIAL APPLICABILITY

The present invention is available for cultivation of protoporphyrinogen IX oxidase inhibitor-resistant crop plants.

1. A method for cultivating a crop plant into which has been introduced either one or both of:
(1) DNA comprising a nucleotide sequence encoding the amino acid sequence of a cytochrome P450 that shows saflufenacil metabolizing activity, and
(2) DNA comprising a nucleotide sequence encoding the amino acid sequence of a protein that shows protoporphyrinogen IX oxidase activity;
wherein said method comprises applying a weed control agent that contains saflufenacil as an active ingredient to an area where said crop plant is cultivated.

2. The cultivation method according to claim 1, wherein said cytochrome P450 is an actinomyces cytochrome P450.

3. The cultivation method according to claim 1, wherein said cytochrome 2450 is selected from the group consisting of:
(1) cytochrome P450 derived from actinomyces belonging to the genus Streptomyces;
(2) cytochrome P450 comprising an amino acid sequence having 90% or more sequence homology with the amino acid sequence of SEQ ID NO: 1 or 2;
(3) cytochrome P450 comprising the amino acid sequence of SEQ ID NO: 1; and
(4) cytochrome P450 comprising the amino acid sequence of SEQ ID NO: 2.

4. The cultivation method according to claim 1, wherein said protein that shows protoporphyrinogen IX oxidase activity is derived from a plant.

5. The cultivation method according to claim 1, wherein said protein that shows protoporphyrinogen IX oxidase activity is selected from the group consisting of:
(1) a protein which is derived from a plant and shows protoporphyrinogen IX oxidase activity that is inhibited by saflufenacil;
(2) a protein which is derived from a plant and shows protoporphyrinogen IX oxidase activity that is not inhibited by saflufenacil;
(3) a protein comprising an amino acid sequence with 90% or more sequence homology with the amino acid sequence of SEQ ID NO: 3 and showing protoporphyrinogen IX oxidase activity; and
(4) a protein comprising the amino acid sequence of SEQ ID NO: 3 and showing protoporphyrinogen IX oxidase activity.

6. A method for screening a cell resistant to a protoporphyrinogen IX oxidase inhibitory-type herbicidal compound, wherein said method comprises:
introducing into and expressing in a plant cell either one or both of;
(1) DNA comprising a nucleotide sequence encoding the amino acid sequence of a cytochrome P450 that shows saflufenacil metabolizing activity, and
(2) DNA comprising a nucleotide sequence encoding the amino acid sequence of a protein that shows protoporphyrinogen IX oxidase activity; and
contacting said plant cell with saflufenacil.

7. The screening method according to claim 6, wherein said cytochrome P450 is an actinomyces cytochrome P450.

8. The screening method according to claim 6, wherein said cytochrome P450 is selected from the group consisting of:
(1) cytochrome P450 derived from actinomyces belonging to the genus Streptomyces;
(2) cytochrome P450 comprising an amino acid sequence having 90% or more sequence homology with the amino acid sequence of SEQ ID NO: 1 or 2;
(3) cytochrome P450 comprising the amino acid sequence of SEQ ID NO: 1; and
(4) cytochrome P450 comprising the amino acid sequence of SEQ ID NO: 2.

9. The screening method according to claim 6, wherein said protein that shows protoporphyrinogen IX oxidase activity is derived from a plant.

10. The screening method according to claim 6, wherein said protein that shows protoporphyrinogen IX oxidase activity is selected from the group consisting of:
(1) a protein which is derived from a plant and shows protoporphyrinogen IX oxidase activity that is inhibited by saflufenacil;
(2) a protein which is derived from a plant and shows protoporphyrinogen IX oxidase activity that is not inhibited by saflufenacil;
(3) a protein comprising an amino acid sequence with 90% or more sequence homology with the amino acid sequence of SEQ ID NO: 3 and showing protoporphyrinogen IX oxidase activity; and
(4) a protein comprising the amino acid sequence of SEQ ID NO: 3 and showing protoporphyrinogen IX oxidase activity.


SEQUENCE LISTING (3 sequences)

SEQ ID NO: 1 - protein, 398 aa, Streptomyses steffisburgensis IFO13446T
MSDTTAPVAFPQSRTCPYHPPAAYEPLRAERPLTRITLFDGREAWLVSGHATARALLADPRLSSDRDRPGFPAPTARFAGIRNRRTALLGVDDPEHRVQRRMVAGDFTLKRAAGLRPRIQRIVDRRLDAMIAQGPPADLVSSFALPVPSMVICALLGVPYADHDFFETQSRRLLRGPQTADVMDARARLDEYFGELIDRKRKEPGAGLLDDLVQRQLRDGALDREGLIALALILLVAGHETTANMISLGTFTLLQHPERLAELRADPRLLPAAVEELMRMLSIADGLLRLAVEDIEVAGTTIRKGDGVVFLTSVINRDETVYPEPDTLDWHRSARHHVAFGFGIHQCLGQNLARAELEIALWTLFDRLPTLRLAAPAEEIAFKPGDTIQGMLELPVTW

SEQ ID NO: 2 - protein, 398 aa, Streptomyses roseorubens IFO13682T
MTDTTAPVAFPQSRTCPYHPPAAYEPLRAERPLTRITLFDGREAWLVSGHATARALLADPRLSSDRDRPGFPTPTARFAGIRNRRTALLGVDDPEHRAQRRMVVGDFTLKRAAALRPRIQRIVDERLDAMIAQGPPADLVSAFALPVPSMVICALLGVPYADHDFFEAQSRRLLRGPGTADVQDARSRLEEYFGELIDRKREDPGTGLLDDLVQRQPGDGGPDREGLIAMALILLVAGHETTANMISLGTFTLLQHPERLAELRADSEVMPAAVEELMRLLSIADGLLRIAVEDVEVAGTTIRAGEGVVFATSVINRDETVFAEPDTLDWSRPARHHVAFGFGIHQCLGQNLARAELEIALGTLFGRLPTLRLAAPPDEIPFKPGDTIQGMLELPVTW

SEQ ID NO: 3 - protein, 543 aa, Glycine max
MVSVFNEILFPPNQTLLRPSLHSPTSFFTSPTRKFPRSRPNPILRCSIAEESTASPPKTRDSAPVDCVVVGGGVSGLCIAQALATKHANANVVVTEARDRVGGNITTMERDGYLWEEGPNSFQPSDPMLTMVVDSGLKDELVLGDPDAPRFVLWNRKLRPVPGKLTDLPFFDLMSIGGKIRAGFGALGIRPPPPGHEESVEEFVRRNLGDEVFERLIEPFCSGVYVGDPSKLSMKAAFGKVWKLEKNGGSIIGGTFKAIQERNGASKPPRDPRLPKPKGQTVGSFRKGLTMLPDAISARLGNKVKLSWKLSSISKLDSGEYSLTYETPEGVVSLQCKTVVLTIPSYVASTLLRPLSAAAADALSKFYYPPVAAVSISYPKEAIRSECLIDGELKGFGQLHPRSQGVETLGTIYSSSLFPNRAPPGRVLLLNYIGGATNTGILSKTDSELVETVDRDLRKILINPNAQDPFVVGVRLWPQAIPQFLVGHLDLLDVAKASIRNTGFEGLFLGGNYVSGVALGRCVEGAYEVAAEVNDFLTNRVYK